(19) United States
(12) Patent Application Publication
(10) Pub. No.: US 2024/0395391 A1
(43) Pub. Date: Nov. 28, 2024

(54) METHOD FOR PROVIDING CONTENT FOR COGNITIVE OR LANGUAGE REHABILITATION TRAINING BASED ON CLOUD SERVER

(71) Applicant: MINDHUB CO., LTD., Anyang-Si (KR)

(72) Inventor: Hae Sung LEE, Anyang-Si (KR)

(73) Assignee: MINDHUB CO., LTD., Anyang-Si (KR)

(21) Appl. No.: 18/745,311

(22) Filed: Jun. 17, 2024

Related U.S. Application Data
(63) Continuation of application No. PCT/KR2022/020626, filed on Dec. 16, 2022.

(30) Foreign Application Priority Data
Dec. 17, 2021 (KR) 10-2021-0181989
Oct. 18, 2022 (KR) 10-2022-0134355

(51) Int. Cl. G16H 20/70; G16H 10/60

(52) U.S. Cl. CPC G16H 20/70; G16H 10/60

(57) ABSTRACT

The present disclosure relates to a method and apparatus for providing content for cognitive or language rehabilitation training based on a cloud server, and may include, upon detecting a login of a specific account into a platform linked to the cloud server, identifying a permission of the specific account; as a result of the identification, based on the specific account being a patient account, collecting at least one medical information for the patient input from at least one connection account linked to the patient account; generating status information of the patient by analyzing the at least one medical information; searching at least one content based on the status information using an artificial intelligence-based learning model; and generating and providing training information using the searched at least one content.

### CROSS-REFERENCE TO RELATED APPLICATIONS

The present application is a continuation of International Patent Application No. PCT/KR2022/020626, filed on Dec. 16, 2022, which is based upon and claims the benefit of priority to Korean Patent Application Nos. 10-2021-0181989 filed on Dec. 17, 2021 and 10-2022-0134355 filed on Oct. 18, 2022. The disclosures of the above-listed applications are hereby incorporated by reference herein in their entirety.

### BACKGROUND

### 1. Technical Field

The present disclosure relates to a method and system for cognitive or language rehabilitation treatment, and more particularly, to a method for providing content for cognitive or language rehabilitation training based on a cloud server.

### 2. Description of Related Art

In general, brain diseases such as stroke and dementia cause impairment of cognitive and language abilities.

A language disorder refers to a delay in language acquisition due to a defect in one of the language chains, such as the series of processes necessary to understand or express spoken language, that is, the input process, cognitive process, and output process. Types of communication disorders that show significant deviations from normal processes are collectively called language disorders.

These language disorders not only cause difficulties in personal communication but also impose many restrictions on social and economic activities, so it is most important to provide accurate prescriptions for them.

Previously, patients and medical personnel (doctors, nurses, rehabilitators, etc.) performed various activities together in offline spaces for the purpose of speech rehabilitation treatment, but this had the limitation of requiring patients and medical personnel to face each other directly in a limited space.

Therefore, in addition to providing speech rehabilitation treatment services that overcome existing face-to-face treatment and enable patients to perform various customized speech rehabilitation treatments at a desired time and place based on a cloud server, there is a need to develop technology that allows continuous service improvement by reflecting opinions of the patient or guardian.

### SUMMARY

The present disclosure is to provide a method and system for providing content for cognitive or language rehabilitation training based on a cloud server that allows a patient to perform various customized rehabilitation training at a desired time and place, but also enables continuous service improvement by reflecting opinions of customers (patients, guardians, medical personnels, therapists, medical institution managers, etc.).

In addition, the present disclosure is to provide a method and system for providing content for cognitive or language rehabilitation training based on a cloud server that provides customized content for rehabilitation training related to at least one or more areas of memory, executive power, attention, auditory comprehension, speech, reading, or writing, but also allows a patient to receive customized speech rehabilitation treatment without restrictions, and allows guardians and/or medical personnel (doctors, nurses, rehabilitators, etc.) to monitor the patient's speech rehabilitation training process, results, status, and the like.

In addition, the present disclosure is to provide a method and system for providing content for cognitive or language rehabilitation training based on a cloud server that provides content enabling rehabilitation training in accordance with a patient's level, such as stage (level), speed, and time, and provides analysis information that analyzes the training pattern and/or training history, such that the patient as well as a guardian, a medical personnel, etc. easily checks the training results, and continuously manages the patient's condition according to rehabilitation training by accumulating and recording them.

The problems to be solved by the present disclosure are not limited to the problems mentioned above, and other problems not mentioned can be clearly understood by those skilled in the art from the description below.

In an aspect of the present disclosure, a method for providing content for cognitive or language rehabilitation training based on a cloud server may include upon detecting a login of a specific account into a platform linked to the cloud server, identifying a permission of the specific account; as a result of the identification, based on the specific account being a patient account, collecting at least one medical information for the patient input from at least one connection account linked to the patient account; generating status information of the patient by analyzing the at least one medical information; searching at least one content based on the status information using an artificial intelligence-based learning model; and generating and providing training information using the searched at least one content, wherein the at least one connection account includes at least one of a guardian account, a therapist account, a medical personnel account, or a medical institution administrator account, wherein the at least one medical information includes at least one of symptom information, diagnosis information, medical personnel information, therapist information, environmental information, or survey response information for the patient, and wherein the status information represents the status of the patient as a quantitative indicator.

In another aspect of the present disclosure, an apparatus for providing content for cognitive or language rehabilitation training based on a cloud server may include a communication module; a storage module configured to store various information for at least one patient, an artificial intelligence-based learning model for providing content for the cognitive or language rehabilitation training, and at least one process; and a control module configured to perform an operation to provide content for the cognitive or language rehabilitation training based on the at least one process, wherein the control muddle is configured to: upon detecting a login of a specific account into a platform linked to the cloud server, identify a permission of the specific account, as a result of the identification, based on the specific account being a patient account, collect at least one medical information for the patient input from at least one connection account linked to the patient account, generate status information of the patient by analyzing the at least one medical information, search at least one content based on the status information using an artificial intelligence-based learning model, and generate and provide training information using the searched at least one content, wherein the at least one connection account includes at least one of a guardian account, a therapist account, a medical personnel account, or a medical institution administrator account, wherein the at least one medical information includes at least one of symptom information, diagnosis information, medical personnel information, therapist information, environmental information, or survey response information for the patient, and wherein the status information represents the status of the patient as a quantitative indicator.

In addition, a computer program stored in a computer-readable recording medium for executing a method for implementing the present disclosure may be further provided.

In addition, a computer-readable recording medium recording a computer program for executing a method for implementing the present disclosure may be further provided.

### BRIEF DESCRIPTION OF THE FIGURES

FIG. 1 is a diagram illustrating a network structure of a system for providing content for cognitive or language rehabilitation training based on a cloud server according to an embodiment of the present disclosure.

FIG. 2 is a diagram illustrating a flow for providing content in a content providing system for cognitive or language rehabilitation training based on a cloud server according to an embodiment of the present disclosure.

FIGS. 3 to 6 are diagrams for explaining the operational configuration of for a content providing system for cognitive or language rehabilitation training based on a cloud server according to an embodiment of the present disclosure.

FIG. 7 is a diagram illustrating the configuration of a content providing device for cognitive or language rehabilitation training based on a cloud server according to an embodiment of the present disclosure.

FIG. 8 is a diagram illustrating an operation for generating training information in a method for providing content for cognitive or language rehabilitation training based on a cloud server according to an embodiment of the present disclosure.

FIG. 9 is a diagram illustrating an operation for generating analysis information in a method for providing content for cognitive or language rehabilitation training based on a cloud server according to an embodiment of the present disclosure.

FIG. 10 is a diagram illustrating a specific operation performed when searching for at least one content in a method of providing content for cognitive or language rehabilitation training based on a cloud server according to an embodiment of the present disclosure.

FIG. 11 is a diagram illustrating a specific operation performed when generating training information is generated in a method of providing content for cognitive or language rehabilitation training based on a cloud server according to an embodiment of the present disclosure.

FIG. 12 is a diagram illustrating an example of patient information stored and managed through a content providing device according to an embodiment of the present disclosure.

FIGS. 13 to 15 are diagrams illustrating an example of a screen on which training performance content according to training information is displayed on the display unit on the patient terminal according to an embodiment of the present disclosure.

FIG. 16 is a diagram illustrating an example of a screen displayed to enable a training setting to perform training through a display unit on a patient terminal according to an embodiment of the present disclosure.

FIG. 17 is a diagram illustrating an example of a screen displayed to enable training to be performed through a display unit on a patient terminal according to an embodiment of the present disclosure.

FIG. 18 is a diagram illustrating an example of a screen displayed to enable confirmation of training result through a display unit on a patient terminal according to an embodiment of the present disclosure.

FIG. 19 is a diagram illustrating an example of a screen where analysis information according to performing training is displayed through a display unit on a patient terminal according to an embodiment of the present disclosure.

FIG. 20 is a diagram illustrating an example of a screen on which training performance history for a specific patient is displayed through a display unit on a patient terminal according to an embodiment of the present disclosure.

FIG. 21 is a diagram illustrating an example of a screen in which training results performed by a specific patient on different days are compared and displayed through a display unit on a patient terminal according to an embodiment of the present disclosure.

FIG. 22 is a diagram illustrating an example of each result report as a result of training performed on different dates according to an embodiment of the present disclosure.

FIG. 23 is a diagram illustrating an example of a screen in which

content for performing attention training through a patient terminal is displayed through a display unit on a patient terminal according to an embodiment of the present disclosure.

FIG. 24 is a diagram illustrating an example of a screen in which content for performing memory training through a patient terminal is displayed through a display unit on a patient terminal according to an embodiment of the present disclosure.

FIG. 25 is a diagram illustrating an example of a screen in which content for performing executive ability training through a patient terminal is displayed through a display unit on a patient terminal according to an embodiment of the present disclosure.

FIGS. 26 to 29 are diagrams illustrating a screen in which content for performing training for each of listening (auditory comprehension), speaking, reading, and writing is displayed through a patient terminal according to an embodiment of the present disclosure.

FIGS. 30 to 33 are diagrams illustrating an example of a graph generated according to an embodiment of the present disclosure.

### DETAILED DESCRIPTION

The advantages and features of the present disclosure, and methods for achieving them, will become clear with reference to the embodiments described in detail below along with the accompanying drawings. However, the present disclosure is not limited to the embodiments disclosed below and may be implemented in various different forms. The present embodiments are merely provided to ensure that the present disclosure is complete, and provided to fully convey the scope of the present disclosure to those skilled in the art to which the present disclosure pertains. The present disclosure is defined only by the scope of the claims.

The terminology used herein is for the purpose of describing embodiments and is not intended to limit the disclosure. As used herein, singular forms also include plural forms, unless specifically stated otherwise in the context. As used in the specification, “comprises” and/or “comprising” does not exclude the presence or addition of one or more other elements in addition to the mentioned elements. The same reference numerals refer to the same elements throughout the specification, and “and/or” includes each and every combination of one or more of the referenced elements. Although “first”, “second”, etc. are used to describe various components, these components are of course not limited by these terms. These terms are merely used to distinguish one component from another. Therefore, the first component mentioned below may also be the second component within the technical spirit of the present disclosure.

Unless otherwise defined, all terms (including technical and scientific terms) used in this specification may be used with meanings commonly understood by those skilled in the art to which this disclosure pertains. Additionally, terms defined in commonly used dictionaries are not to be interpreted ideally or excessively unless clearly specifically defined.

In the drawings, the same reference numeral refers to the same element. This disclosure does not describe all elements of the embodiments, and general content or overlapping content between embodiments in the technical field to which this disclosure pertains is omitted. As used in the specification, the term “unit” or “module” refers to a hardware component such as software, FPGA, or ASIC, and the “unit” or “module” performs certain roles. However, “part” or “module” is not limited to software or hardware. A “unit” or “module” may be configured to reside on an addressable storage medium and may be configured to run on one or more processors. Thus, as an example, a “part” or “module” refers to components such as software components, object-oriented software components, class components, and task components, processes, functions, properties, procedures, subroutines, segments of program code, drivers, firmware, microcode, circuits, data, databases, data structures, tables, arrays, and variables. The functionality provided within components and “parts” or “modules” may be combined into smaller components and “parts” or “modules” or into additional components, or further separated into “parts” or “modules”.

Throughout this specification, when a part is referred to as being “connected” to another part, this includes “direct connection” and “indirect connection”, and the indirect connection may include connection via a wireless communication network.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

The terms used in the following description are defined as follows.

In describing the present disclosure, a device is limited to a content providing device 100, but may further include a server, a computer, and/or a portable terminal, or may take the form of any one of them.

Here, the computer may include, for example, a laptop equipped with a web browser, a desktop, a laptop, a tablet PC, a slate PC, and the like.

The server device is a server that processes information by communicating with external devices, and may include an application server, a computing server, a database server, a file server, a mail server, a proxy server, a web server, and the like.

The portable terminal is, for example, a wireless communication device that guarantees portability and mobility, and may include all types of handheld wireless communication devices such as PCS (Personal Communication System), GSM (Global System for Mobile communications), PDC (Personal Digital Cellular), and PHS (Personal Handyphone System)), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, a smart phone, and the like and wearable devices such as watches, rings, bracelets, anklets, necklaces, glasses, contact lenses, head-mounted devices (HMD), or the like.

Hereinafter, the operational principles and embodiments of the present disclosure are described with reference to the accompanying drawings.

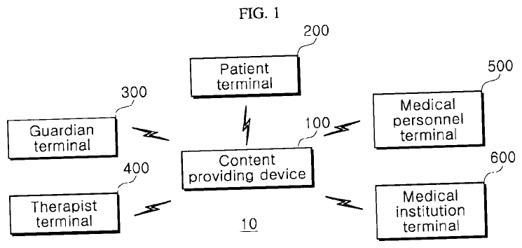

FIG. 1 is a diagram illustrating a network structure of a system for providing content for cognitive or language rehabilitation training based on a cloud server according to an embodiment of the present disclosure.

Referring to FIG. 1, a system for providing content (hereinafter referred to as ‘content providing system’) 10 for cognitive or language rehabilitation training based on a cloud server according to an embodiment of the present disclosure may include a content providing device 100, a patient terminal 200, a guardian terminal 300, a therapist terminal 400, a medical personnel terminal 500, and a medical institution terminal 600.

The content providing device 100 is a device for providing a content providing service for rehabilitation training in the content providing system 10, and may be connected to at least one of the patient terminal 200, the guardian terminal 300, the therapist terminal 400, the personnel terminal 500, or the medical institution terminal 600 based on wired or wireless communication. In this case, the content providing device 100 may be a server, but is not limited thereto.

The content providing device 100 provides a content providing service based on a platform linked to a cloud server, and performs a subscription service for a patient, a guardian, a therapist, a medical personnel, and/or a medical institution, which are intended to receive the content providing service. In this case, when the patient, the guardian, the therapist, the medical personnel, and/or the medical institution signs up, their respective accounts and permissions are set (created), and the content providing device 100 may further perform a linkage process between the related accounts among the respective accounts. As a result, content providing services may be provided through the platform through each of the patient terminal 200, the guardian terminal 300, the therapist terminal 400, the medical personnel terminal 500, and the medical institution terminal 600. However, as different permissions are set for each terminal, the contents of the services provided may be different. At this time, each permission may be set by a service provider and/or a medical personnel of the content providing device 100.

Specifically, upon detecting a login of a specific account into a platform linked to the cloud server, the content providing device 100 identifies a permission of the specific account. As a result of the identification, based on the specific account being a patient account, the content providing device 100 collects at least one medical information for the patient input from at least one connection account linked to the patient account. In this case, the at least one connection account includes at least one of a guardian account, a therapist account, a medical personnel account, or a medical institution administrator account. In addition, the medical information may include at least one of medical data, treatment data, symptom information, diagnosis information, medical personnel information, therapist information, environmental information, or survey response information for the patient, each included in the medical information, and the data or information may be input (obtained) from at least one of the patient terminal 200, the guardian terminal 300, the therapist terminal 400, the medical personnel terminal 500, or the medical institution terminal 600. Here, the symptom information includes at least one piece of information about the patient's symptoms confirmed during treatment, and the diagnosis information may include at least one information which is determined and entered by a medical staff through the symptom information, such as the name of the diagnosis, a level of understanding, a level of expression, and a level of cognitive ability.

Thereafter, the content providing device 100 generates status information of the patient by analyzing the at least one medical information, and searches at least one content based on the status information. Meanwhile, the content providing device 100 generates training information for the patient using the at least one searched content and then provides the generated training information through the terminal to which the specific account is logged in, or provides the generated training information through patient terminal 200 corresponding to the specific account by an additional request. At this time, when providing training information, the content providing device 100 may perform a separate payment procedure, automatically performing payment using pre-stored payment information, or performing payment by manually entering payment information. Here, the at least one preset terminal may include at least one of the patient terminal 200, the guardian terminal 300, the therapist terminal 400, the medical personnel terminal 500, or the medical institution terminal 600 of the patient.

At this time, when training result information generated as the patient performs training through the corresponding terminal to which the training information is provided is received from the terminal, the content providing device 100 generates the training result information by analyzing analysis information for the patient, and then, transmits the analysis information to at least one preset terminal. The training result information may store (record) the degree of training performance for the cognitive domain or language domain, training ratio, training domain, and accuracy for each training domain by date. Whenever each training is performed through the patient account, as the training result information is accumulated and stored, the analysis information for the patient analyzed based on it may also be accumulated and stored or updated.

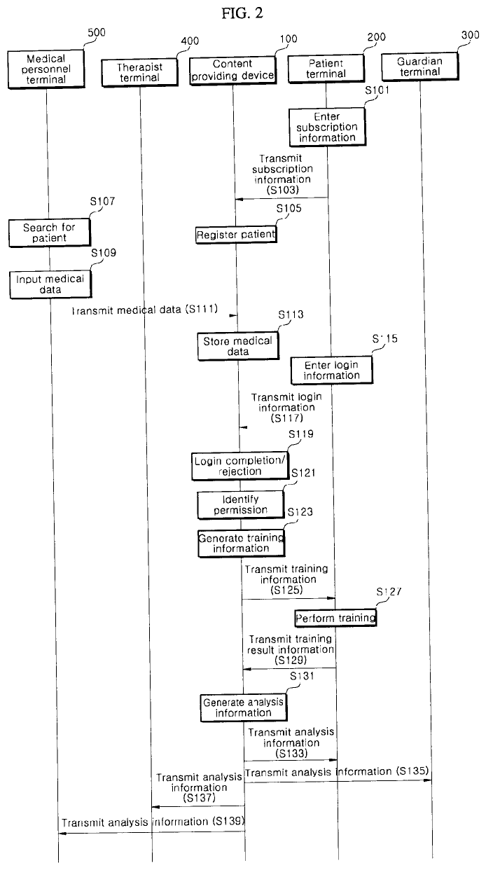

As described above, the content providing device 100 may be provided with an artificial intelligence-based learning model to generate the status information, the training information, and the analysis information. In this case, the learning model may be individually provided for each information. The operation of generating each piece of information will be described in detail below based on FIGS. 2 to 11.

Meanwhile, as a result of previously identifying the permission of the specific account, in the case that the specific account is any one of the guardian account, the therapist account, the medical personnel account, and the medical institution account, the content providing device 100 identifies at least one patient account linked to the identified account, and when one patient account is selected by the user of the identified account, provides the analysis information corresponding to the patient account. In this case, the analysis information may be generated by a user of the account (i.e., any one of a guardian, a therapist, a medical personnel, or medical institution administrator) using the own terminal (i.e., one of the guardian terminal 300, the therapist terminal 400, the medical personnel terminal 500, and the medical institution terminal 600) based on a setting condition (including training date, training time, training area, etc.). For example, the analysis information containing a graph and/or an accuracy for each cognitive area and/or language area performed in a date range (period) set according to the setting condition may be generated.

Meanwhile, the content providing device 100 may further grant editing permission for patient information to each of the therapist account, the medical personnel account, and the medical institution account, and in this case, the editing permission may be different from each other. For example, the therapist may add more content to perform more reinforcement training or exclude content that is deemed unnecessary from the patient's training information, and after checking the training result information, perform an opinion input (including treatment data) therefor and/or filling out a survey for the therapist. In addition, the medical personnel (medical institution administrator) may be granted the permission including the permission of the therapist, to perform the above additional operations, and after treating the patient, the permission of inputting at least one of the medical data, symptom information, and diagnosis information (the symptom information, the diagnosis information, etc. may be included in the medical data), and after checking the analysis information, entering opinion on it and/or filling out a survey for the medical personnel (medical institution).

Meanwhile, the patient terminal 200 is the patient's terminal, and the patient uses the patient terminal 200 to receive the content providing service from the content providing device 100 based on a platform linked to a cloud server. Specifically, the training information is received from the content providing device 100 based on the platform through the patient terminal 200, training is performed on the training information, and training result information is transmitted to the content providing device 100. Thereafter, when the analysis information generated based on the training result information is received from the content providing device 100 in response, this may be displayed on a display unit of the patient terminal 200 for the patient to check.

Meanwhile, the guardian terminal 300, the therapist terminal 400, and the medical personnel terminal 500 are terminals used by guardians, therapists, and medical personnels for a specific patient (i.e., linked to the specific patient's account), respectively. and the medical institution terminal 600 may include at least one of a terminal provided in the medical institution or a terminal used by the administrator of the medical institution. Each user may view or edit (including create, input, and delete) various information about a specific patient from the content providing device 100 through the guardian terminal 300, the therapist terminal 400, the medical personnel terminal 500, and the medical institution terminal 600 or receive various information and/or notifications from the content providing device 100. At this time, various information and/or notifications may be transmitted only to at least one preset terminal, so even guardians, therapists, medical personnel, and medical institutions for that specific patient may not receive the various information and/or notifications.

In addition, on the display unit of each of the guardian terminal 300, the therapist terminal 400, the medical personnel terminal 500, and the medical institution terminal 600, a template including a form for entering data or information may be displayed differently depending on the permission, and the template may be changed or edited by an administrator of the content providing device 100.

Previously, in describing the present disclosure, the description was limitedly described to the case where a specific patient receives the training information through the patient terminal 200 and performs the training, but the specific patient may also receive the training information through another terminal and perform the training through the terminal. For example, when a specific patient logs in through a terminal provided at a medical institution, that is, the medical institution terminal 600, the content providing device 100 determines whether it is a patient account or a medical institution account through the login information, and provide different training information depending on the identified account. That is, when generating training information, the content providing device 100 may generate the training information differently depending on the type of terminal on which the login is performed, that is, whether it is the patient terminal 200 or another terminal (the guardian terminal 300, the therapist terminal 400, the medical personnel terminal 500, the medical institution terminal 600, or other terminals).

Meanwhile, each of the patient terminal 200, the guardian terminal 300, the therapist terminal 400, the medical personnel terminal 500, and the medical institution terminal 600 may be a computer, an UMPC (Ultra Mobile PC), a workstation, a net-book, a PDA (Personal Digital Assistant), a portable computer, a web tablet, and a wireless phone, a mobile phone, a smart phone, a pad, a smart watch, a wearable terminal, an e-book, a PMP (portable multimedia), a portable game console, a navigation device, a black box or digital camera, or other mobile communication terminal, on which a plurality of applications desired by each user may be installed and executed.

That is, each of the patient terminal 200, the guardian terminal 300, the therapist terminal 400, the medical personnel terminal 500, and the medical institution terminal 600 may be installed with a separate application or program or provided with the content providing service from the content providing device 100 through the platform.

However, FIG. 1 is merely an embodiment for convenience of explanation, and the patient terminal 200, the guardian terminal 300, the therapist terminal 400, the medical personnel terminal 500, and the medical institution terminal 600 correspond to the user's terminal for receiving the service provided by the content providing system 10, and a terminal of other authorized institution or other personnel may be further included, and the type (model) and number of the terminal is not limited. That is, the patient terminal 200, the guardian terminal 300, the therapist terminal 400, the medical personnel terminal 500, and/or the medical institution terminal 600 may be plural, and each of the terminals may be different from each other, or some or all of the terminals may be the same model.

FIG. 2 is a flowchart illustrating a method of providing prediction information by artificial intelligence-based data analysis according to an embodiment of the present disclosure, in which a specific patient performs training from the content providing device 100 through the patient terminal 200.

Referring to FIG. 2, the specific patient may perform a subscription process through the content providing device 100 to receive a content provision service. To this end, the specific patient executes the platform through the patient terminal 200, enters subscription information into the template for subscription (step S101), and transmits the subscription information to the content providing device 100 (step S103).

Next, the content providing device 100 registers a new patient based on the subscription information received from the patient terminal 200 (step S105). Here, the subscription information includes basic information and account information (including at least one of the patient account, the guardian account, the therapist account, or the medical personnel account) for the specific patient. For example, the subscription information may include at least one of the name of the specific patient, the patient number, the date of birth, the gender, the residential address, the current status, the registration date, the identifier (ID), or the password.

At this time, the operation of registering a new patient includes the operation of adding the specific patient as a target of service provider and storing the subscription information, as well as connecting at least one of the patient account, the guardian account, the therapist account, or the medical personnel account related to the specific patient. However, this is also just one embodiment, and the operation of connecting each account related to the specific patient may be performed as each account requests connection to the content providing device 100.

After the medical staff performs treatment on the specific patient, the medical personnel searches for the specific patient (step S107) by running the platform through the medical personnel terminal 500, and when medical data is input through the medical personnel terminal 500 as one of the medical information included in the patient information of the searched specific patient (step S109), transmits the medical data to the content providing device 100 (step S111).

Next, when the medical data is received by the medical personnel terminal 500, the content providing device 100 stores the medical data by including it in the patient information (step S113).

Thereafter, when the patient executes the platform and enters login information (step S115), the patient terminal 200 transmits the login information to the content providing device 100 (step S117).

Next, the content providing device 100 processes login completion or login rejection based on the login information received from the patient terminal 200 (step S119). Although not shown in FIG. 2, if login is rejected, the content providing device 100 may transmit a rejection notification to the patient terminal 200.

If the login is completed, the content providing device 100 verifies the account based on the login information and identifies the permission (checks whether it is the patient account) (step S121), after generating training information using the patient information corresponding to the patient account (step S123), and transmits the training information to the patient terminal 200 (step S125).

Afterwards, when the patient performs training as the patient terminal 200 displays the training information on the display unit (step S127), the patient terminal 200 transmits the training result information to the content providing device 100 (step S129).

Next, the content providing device 100 stores the training result information received from the patient terminal 200, generates analysis information using the training information (step S131), and then transmits the analysis information to at least one preset terminal (steps S133 to S139). FIG. 2 shows a case where the patient terminal 200, the guardian terminal 300, the therapist terminal 400, and the medical personnel terminal 500 connected to the patient account are set as at least one terminal. Depending on the settings, the analysis information is transmitted to the respective terminal, but this corresponds to merely one embodiment. In other words, the analysis information may not be transmitted to some of the terminals connected to the patient account, and this may be changed and set if the terminal has the setting (editing) permission. In addition, although FIG. 2 shows that the analysis information is transmitted sequentially, this is also just one embodiment and may be transmitted simultaneously, and the transmission form and/or the transmission order are not limited.

Meanwhile, although not shown in FIG. 2, after the therapist performs training with the patient based on training information or after the analysis information is received through the therapist terminal 400, the therapist may execute the platform through the therapist terminal 400 and enter the result of training (i.e., training result information) or the opinion (including treatment data) on the analysis information, or conduct a survey of therapists related to the training result information or the analysis information and transmit the survey results, and further, edit and/or create the training information to include reinforcement training.

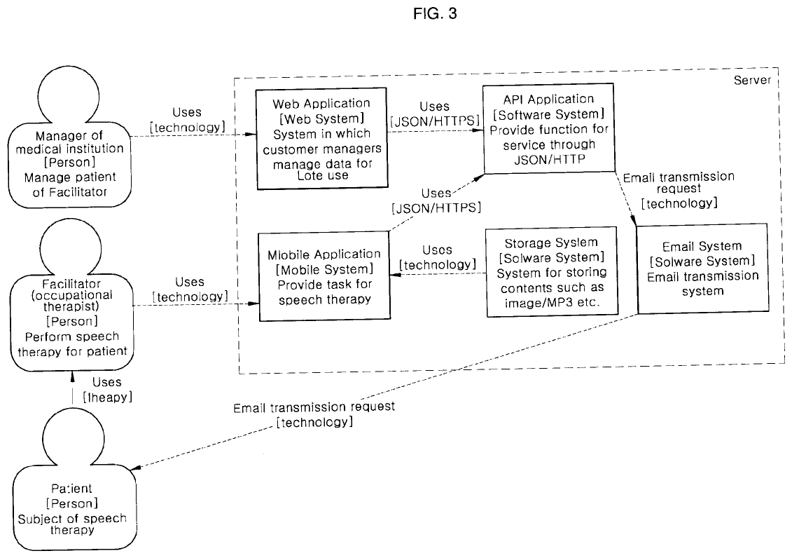

FIGS. 3 to 6 are diagrams for explaining the operational configuration of for a content providing system for cognitive or language rehabilitation training based on a cloud server according to an embodiment of the present disclosure.

In the content providing system 10 according to an embodiment of the present disclosure, the content providing device allows an access of the patient, the guardian, the therapist (or Facilitator), the medical personnel (doctor in charge, nurse in charge, or rehabilitation staff), and the administrator of the medical institution based on their respective permissions through a terminal application (mobile application) or a web application provided on a website. Accordingly, the content providing device 100 performs the management of the information of the patient, the guardian, the therapist (or Facilitator), the medical personnel (doctor in charge, nurse in charge, or rehabilitation staff), and the medical institution related to the patient as well as the patient management, the task provision, the content creation, and the content management.

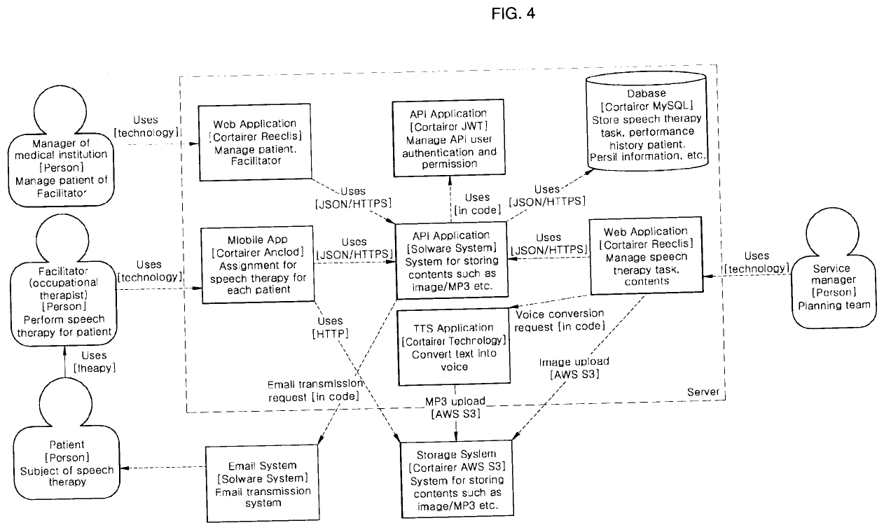

Referring to FIG. 3, the medical personnel or the manager of medical institution provides functions for the content providing service in the terminal application (mobile application) or the web application provided on a website, and the therapist provides tasks (or additional tasks) for training and provides the training information to the patient subject to training through the content providing device 100. In more detail, referring to FIG. 4, the medical personnel manages at least one patient and/or therapist (or Facilitator) through the medical personnel terminal 500, and manages API user authentication and permission. Meanwhile, based on the training tasks for each patient provided by the therapist through the therapist terminal 400, the service provider of the content providing device 100 manages the training tasks and content, and each patient may be provided with the training information including at least one content for training by executing the platform through the respective patient terminal 200, but provided with the training information through e-mail, SNS, or SMS.

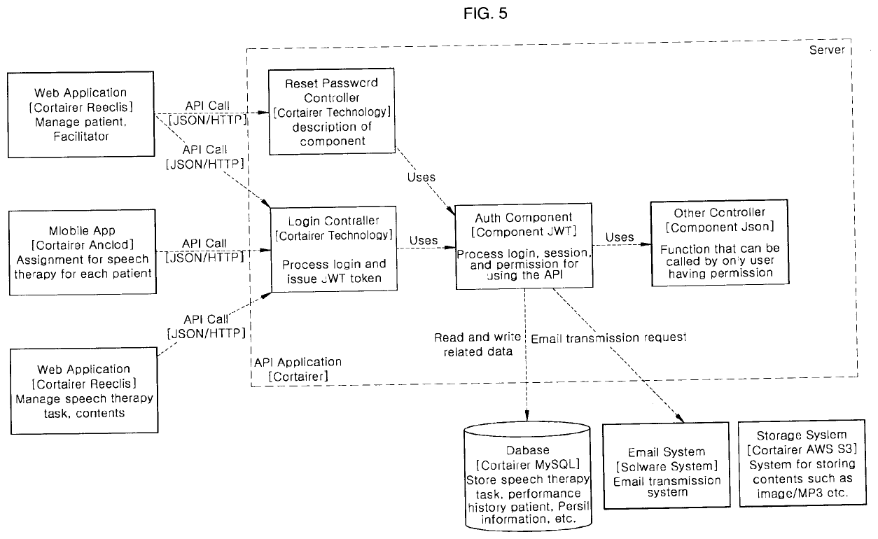

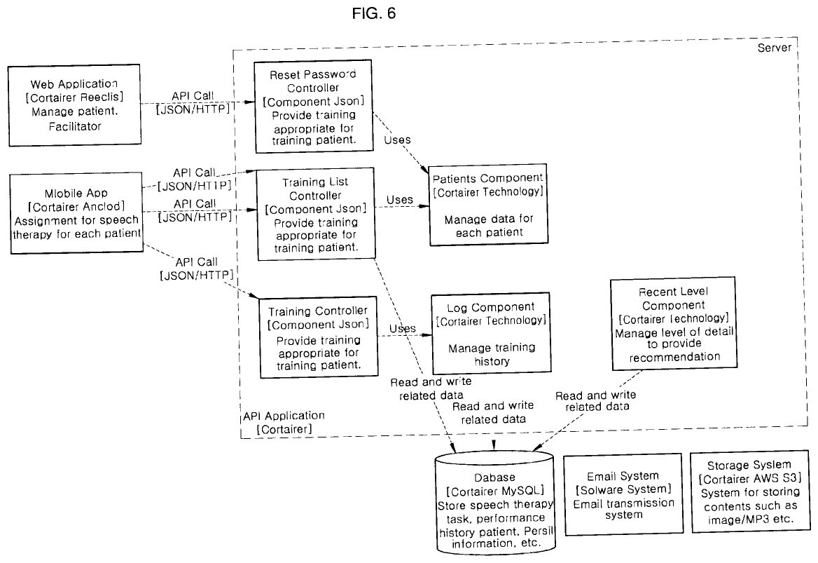

Referring to FIGS. 5 and 6, the content providing device 100 manages at least one patient, training tasks, and content for each patient through the mobile application and the web application. In addition, the content providing device 100 may provide appropriate training to each patient, manage information, data, and training history for each patient, and manage the level of detailed items to provide content recommendations.

Previously, in describing FIGS. 3 to 6, the training task may be a task for performing at least one of cognitive therapy or speech therapy, and may further include tasks in which the cognitive therapy and speech therapy are linked to each other.

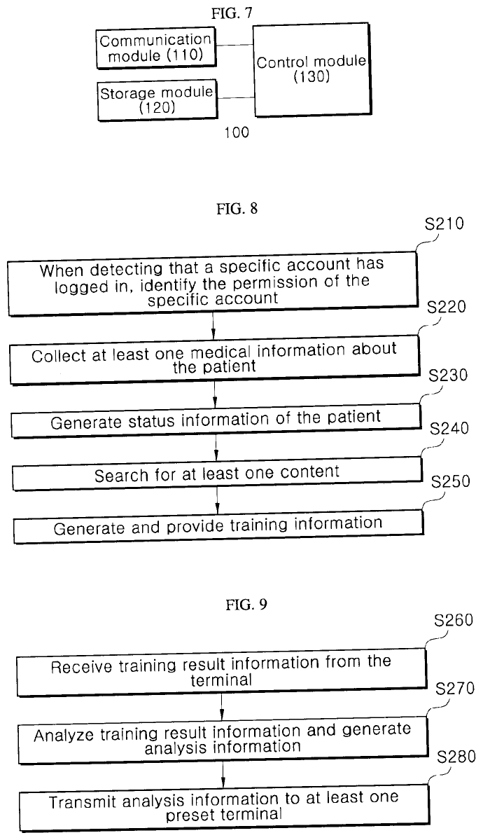

FIG. 7 is a diagram illustrating the configuration of a content providing device for cognitive or language rehabilitation training based on a cloud server according to an embodiment of the present disclosure.

Referring to FIG. 7, the content providing device 100 according to an embodiment of the present disclosure may include a communication module 110, a storage module 120, and a control module 130.

The communication module 110 performs wired or wireless communication with at least one external device (server, etc.). Particularly, when performing wireless communication, the communication module 110 transmits and receives wireless signals in a communication network based on wireless Internet technologies.

The communication module 110 may include one or more components that enable communication with an external device, and may include, for example, at least one of a wired communication module, a wireless communication module, or a short-range communication module, and transmits and receives signals based on this.

Here, the wired communication module is not only a variety of wired communication modules, such as a local area network (LAN) module, a wide area network (WAN) module, or a value added network (VAN) module, but also a variety of cable communication modules, such as Universal Serial Bus (USB), High Definition Multimedia Interface (HDMI), Digital Visual Interface (DVI), recommended standard (RS-232), power line communication, or plain old telephone service (POTS).

The wireless communication module may include a wireless communication module that supports various wireless communication schemes such as GSM (global System for Mobile Communication), CDMA (Code Division Multiple Access), WCDMA (Wideband Code Division Multiple Access), UMTS (universal mobile telecommunications system), TDMA (Time Division Multiple Access), WLAN (Wireless LAN), DLNA (Digital Living Network Alliance), WiBro (Wireless Broadband), WiMAX (World Interoperability for Microwave Access), HSDPA (High Speed Downlink Packet Access), HSUPA (High Speed Uplink Packet Access), LTE (Long Term Evolution), 4G, 5G, 6G, and the like, in addition to the Wi-Fi module and the WiBro (Wireless Broadband).

The short-range communication module is for short range communication, and may support the short range communication using at least one of Bluetooth, RFID (Radio Frequency Identification), Infrared Data Association (IrDA), UWB (Ultra-Wideband), ZigBee, NFC (Near Field Communication), Wi-Fi (Wireless-Fidelity), Wi-Fi Direct, or Wireless USB (Wireless Universal Serial Bus) technologies.

Specifically, the communication module 110 transmits and receives various information to/from at least one of the patient terminal 200, the guardian terminal 300, the therapist terminal 400, the medical personnel terminal 500, or the medical institution terminal 600 based on wired or wireless communication. That is, subscription information, login information, medical treatment data, treatment data, training result information, and the like may be received, and training information, analysis information, and the like may be transmitted through the communication module 110.

The storage module 120 stores data and/or various information supporting various functions of the content providing device 100. A plurality of application programs (or applications) running on the content providing device 100, data and commands may be stored. At least some of these applications may be downloaded from an external server via wireless communication. Meanwhile, the application program may be stored in at least one memory provided in the storage module 120, installed on the content providing device 100, and operated (or functioned) by at least one processor provided in the control module 130.

Meanwhile, at least one memory may include at least one type of storage medium including be a flash memory type, a hard disk type, a multimedia card micro type, or a card type memory (e.g., SD or XD memory, etc.), RAM (Random Access Memory), SRAM (Static Random Access Memory), ROM (Read-Only Memory, ROM), EEPROM (Electrically Erasable Programmable Read-Only Memory), PROM (Programmable Read-Only Memory), magnetic disk, and optical disk. In addition, the memory may store information temporarily, permanently, or semi-permanently, and may be provided as a built-in or removable type.

The storage module 120 may be provided by building a database for providing content for cognitive or language rehabilitation training based on a cloud server, and the database may contain information (including at least one of symptom information, diagnosis information, medical data, treatment data, training history information, and/or additional information, etc.) for each patient subscribed to the content providing service. For this purpose, the storage module 120 may be linked to the cloud server.

The control module 130 may include at least one processor and control all components within the content providing device 100 to process input or output signals, data, information, and the like, perform various processes by executing stored commands, algorithms, and applications, provide predictive information through data analysis using artificial intelligence, or provide or process appropriate information or functions to manage the database.

That is, the control module 130 controls all operations performed by the content providing device 300 described above based on FIGS. 1 and 2.

Specifically, when the control module 130 detects the login of a specific account to the platform linked to the cloud server, control module 130 identifies the permission of the specific account, and generates and provides one of available information according to the identification result.

For example, when the control module 130 identifies that the specific account is a patient account as a result of the identification, the control module 130 collects at least one piece of information for the patient input from at least one connection account linked to the patient account and generates status information for the patient by analyzing the at least one information. Thereafter, the control module 130 searches for at least one content based on the status information using an artificial intelligence-based learning model, and generates training information using the searched at least one content to provide the patient terminal 200, and accordingly, the content providing service may be provided to the patient.

Meanwhile, when training result information is received from the patient terminal 200 in response to the training information, the control module 130 controls to generate analysis information based on the training result information to transmit the analysis information to at least one preset terminal (including at least one of the guardian terminal 300, the therapist terminal 400, the medical personnel terminal 500, and the medical institution terminal 600) among terminals connected to the patient account.

Meanwhile, when the control module 130 confirms that the specific account is one of a guardian account, a therapist account, a medical personnel account, and a medical institution account, the control module 130 controls to provide medical information of the patient searched and selected by the account and perform editing operations such as inputting or deleting additional information or data. At this time, the provided medical information may include different information or data depending on the permission of the account. In other words, instead of providing all of the patient's medical information, only some permitted information/data may be provided according to the permission.

FIG. 8 is a diagram illustrating an operation for generating training information in a method for providing content for cognitive or language rehabilitation training based on a cloud server according to an embodiment of the present disclosure.

Referring to FIG. 8, when the content providing device 100 detects that a specific account has logged in to the platform, the content providing device 100 identifies the permission of the specific account (step S210). As a result of the identification, in the case that the specific account is a patient account, the content providing device 100 collects at least one medical information about the patient input from at least one connection account linked to the patient account (step S220).

Afterwards, the content providing device 100 generates status information of the patient by analyzing at least one medical information collected in step S220 (step S230) and searches for at least one content based on the status information (step S240).

Thereafter, the content providing device 100 generates training information for the patient using the searched at least one content, and transmits and provides the generated training information to the terminal on which the login is performed or the terminal designated (selected) by the patient (step S250).

Meanwhile, although not shown in FIG. 8, after generating the training information in step S250, an operation of performing a payment may be further performed before providing the training information to the terminal to which the specific account is logged in.

FIG. 9 is a diagram illustrating an operation for generating analysis information in a method for providing content for cognitive or language rehabilitation training based on a cloud server according to an embodiment of the present disclosure.

Referring to FIG. 9, when training result information generated as the patient performs training is received from the terminal to which the training information is provided (step S260), the content providing device 100 analyzes the training result information and generates analysis information for the patient (step S270), and then transmits the analysis information to at least one preset terminal (step S280).

In describing FIGS. 8 and 9, overlapping content due to the previous description of FIGS. 1 to 7 has been omitted.

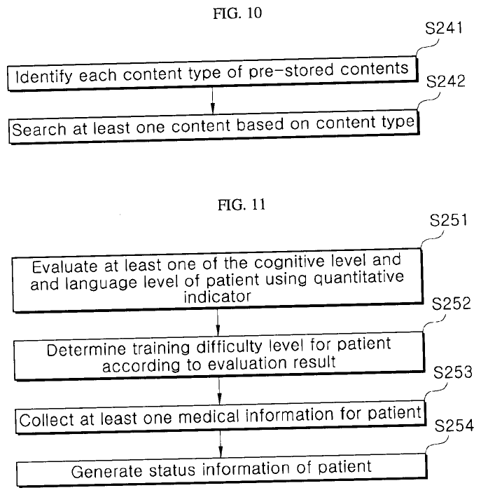

FIG. 10 is a diagram illustrating a specific operation performed when searching for at least one content in a method of providing content for cognitive or language rehabilitation training based on a cloud server according to an embodiment of the present disclosure, and shows the detailed operation of step S240 shown in FIG. 8.

Referring to FIG. 10, the content providing device 100 identifies each content type based on tag information of contents previously stored on a cloud server through big data analysis (step S241), and searches at least one content based on the identified content type (step S242).

Here, the tag information includes detailed subcategory areas, so that content for language rehabilitation training more suitable for individual patients is provided. That is, the content providing device 100 may identify the patient's vulnerabilities in the subcategory area among the tag information and search for content for language rehabilitation training including the tag information to reinforce the subcategory area. Specifically, the content for language rehabilitation training includes tag information of #memory, #execution, and #attention in the cognitive rehabilitation category area, and #auditory comprehension, #speaking, #reading, and #writing in the language rehabilitation category area. For example, when a patient clicks on the cognitive rehabilitation category area for language rehabilitation training, the content for language rehabilitation training including the tag information of #memory and #attention may be placed at the top to improve memory and attention.

Meanwhile, the pre-stored contents may be accumulated and stored by being transmitted from at least one of the content collected based on big data and/or at least one of the therapist terminal 400, the medical personnel terminal 500, and the medical institution terminal 600. For example, the pre-stored contents may include at least one of cognitive therapy content, first speech therapy content, or second speech therapy content. Here, the first speech therapy content may be content that is linked to cognitive therapy and usable without a therapist, and the second speech therapy content may be content that may be performed offline or online through a therapist.

This is to search for at least one content that needs to be performed according to the status information of the patient, and this operation may be performed by either one of at least one learning model provided in the content providing device 100.

FIG. 11 is a diagram illustrating a specific operation performed when generating training information is generated in a method of providing content for cognitive or language rehabilitation training based on a cloud server according to an embodiment of the present disclosure.

Referring to FIG. 11, the content providing device 100 evaluates at least one of the cognitive level and language level of the patient using pre-stored quantitative indicators (step S251), and according to the evaluation result, determines a training difficulty level for the patient (step S252).

Next, the content providing device 100 selects at least one content corresponding to the determined training difficulty among the at least one content searched in step S250 (step S253), and then generates training information by determining a training order for the selected at least one content (step S254).

In other words, as the selected at least one content is displayed on the display unit of the terminal in accordance with the training order determined in step S254, a user may perform training in the arranged order.

However, this is merely one embodiment, and step S254 may be omitted. In this case, as the selected at least one content is displayed in a random order on the display unit of the terminal, a user may perform training in a desired order.

In describing FIG. 7 above, although the content providing device 100 is limited to including only the communication module 110, the storage module 120, and the control module 130, this is for convenience of description, and the component may further include modules classified according to each operation for providing the content providing service. For example, an evaluation module for step S251, a content selection module for step S253, an analysis module for step S270, and the like may be further included. Additionally, the name is also not limited.

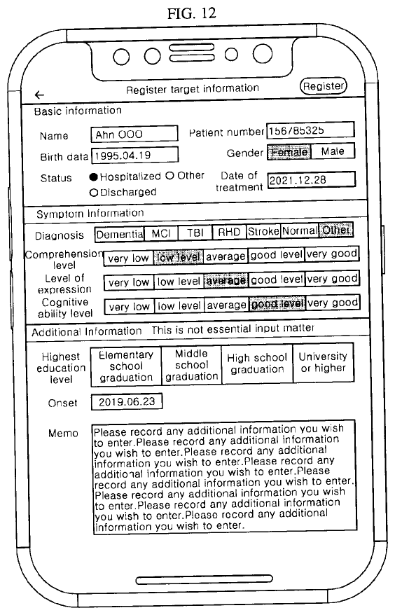

FIG. 12 is a diagram illustrating an example of patient information stored and managed through a content providing device according to an embodiment of the present disclosure.

Referring to FIG. 12, the content providing device 100 according to an embodiment of the present disclosure builds a database by storing (registering) patient information for each patient. In this case, the patient information may include basic information, symptom information, and additional information of the patient.

Here, the basic information may be information input when the patient subscribes to the content providing service provided by the content providing device 100 through the platform. In addition, the symptom information may include the diagnosis, understanding level, expression level, and level of cognitive ability for the patient, and may further include additional information (memos). Here, the symptom information may be information entered according to the permission of medical personnel and therapists capable of medical diagnosis (judgment), but the additional information may be entered by at least one of the patient, the guardian, the therapist, the medical personnel, and the administrator of the medical institution through the platform.

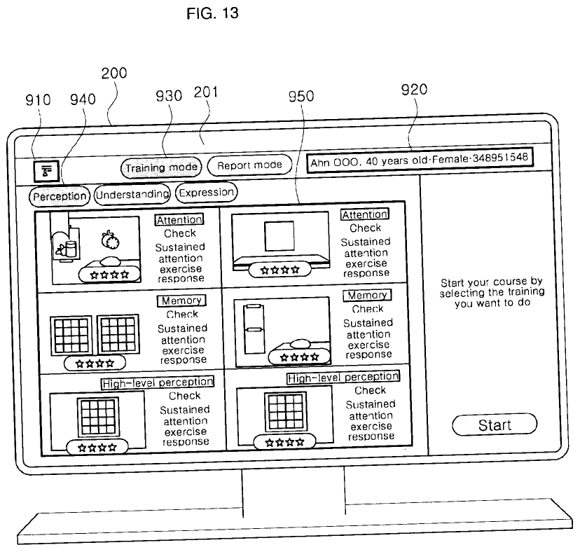

As an example, the patient's language ability is evaluated in each rehabilitation field within the language rehabilitation category (cognitive rehabilitation category, comprehension rehabilitation category, and expression rehabilitation category) based on the basic information, the symptom information, and the additional information about the patient, and the content for language rehabilitation training may be recommended and provided. Hereinafter, an example in which training information for providing content for patient-customized speech rehabilitation training is provided based on FIGS. 13 to 15 will be described.

FIGS. 13 to 15 are diagrams illustrating an example of a screen on which training performance content according to training information is displayed on the display unit 201 on the patient terminal 200 according to an embodiment of the present disclosure.

Referring to FIG. 13, the content providing device 100 provides a training mode 930 for providing content for customized speech rehabilitation training to the patient through the platform. This training mode 930 may be provided according to the fields of cognitive rehabilitation category and language rehabilitation category. In addition, the training mode 930 may provide patient list information 910 and patient profile information 920, and provide training content according to a cognitive rehabilitation category 940, that is, a list 950 consisting of content for speech rehabilitation training. As shown in FIG. 13, the content for language rehabilitation training provided in the cognitive rehabilitation category 940 includes tag information of #attention, #memory, #calculation, and #thinking, respectively, and provides types of training corresponding to each tag information.

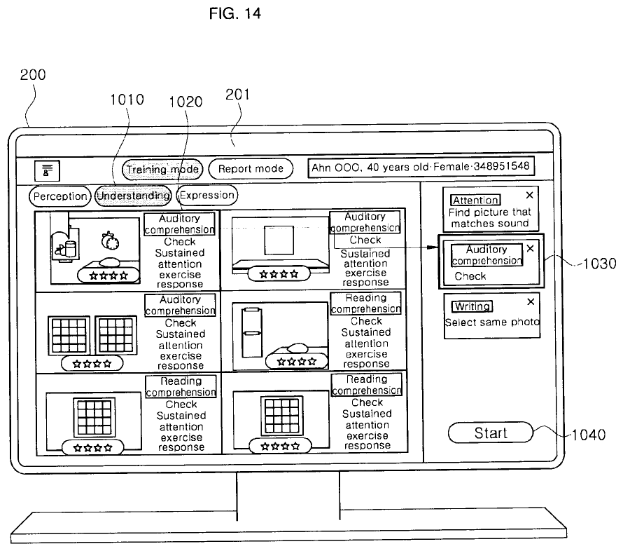

Referring to FIG. 14, a comprehension rehabilitation category 1010 also provides a list of content for speech rehabilitation training, and the contents for speech rehabilitation training provided in the comprehension rehabilitation category 1010 includes tag information of #auditory comprehension and #reading comprehension, respectively. Among the list consisting of content for language rehabilitation training, each training content box 1020 is marked with a content preview photo, name, and difficulty level (training difficulty level of the content). For example, when a patient selects (checks, touches, or clicks) a training content box, a selected training content 1030 may be provided on the right tab bar. At this time, the selected training content may be displayed together with a name/area exposure and a deselection button. When a plurality of training contents is selected, the selected plurality of training contents may be listed and displayed on the tab bar. Meanwhile, when the patient presses a start button 1040, training for at least one content selected by the patient begins.

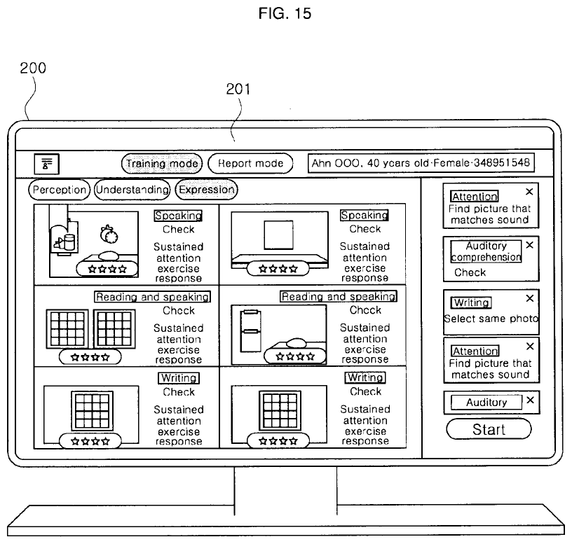

Referring to FIG. 15, information on the expressive rehabilitation category is provided, and the expressive rehabilitation category also provides a list including content for speech rehabilitation training, and the content for speech rehabilitation training provided in the expressive rehabilitation category may contain tag information for #speaking, #reading and speaking, and #writing. The list and tab bar composed of content for language rehabilitation training as described above may be supported in all training modes.

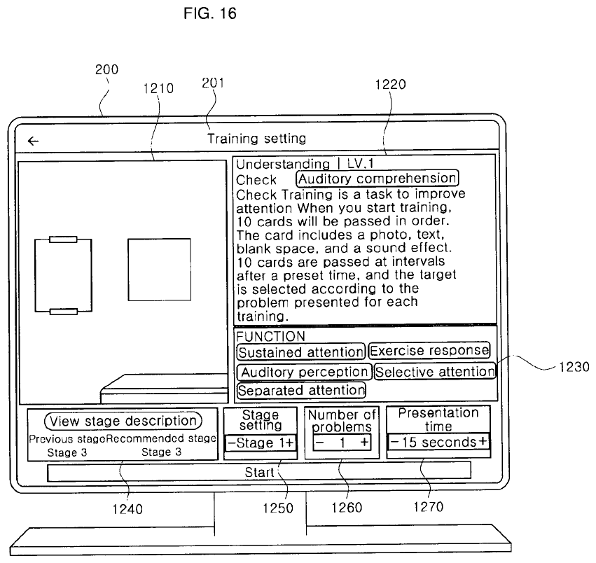

FIG. 16 is a diagram illustrating an example of a screen displayed to enable a training setting to perform training through a display unit on a patient terminal according to an embodiment of the present disclosure. The screen for setting training may be displayed on the display unit 201 of the patient terminal 200 by selecting by checking, touching, or clicking ‘Start button’ on the tab bar that lists one training content from the list of content for speech rehabilitation training.

Referring to FIG. 16, a training content preview photograph 1210 and a training field description 1220 including a difficulty level, a content name, and training description may be provided, and a training keyword 1230 applied among 8 improvement areas may be provided together with a stage description 1240, a stage setting 1250, the number of problems 1260, and a presentation time setting 1270. Accordingly, when the ‘Start button’ is pressed, training on the content selected by the patient begins.

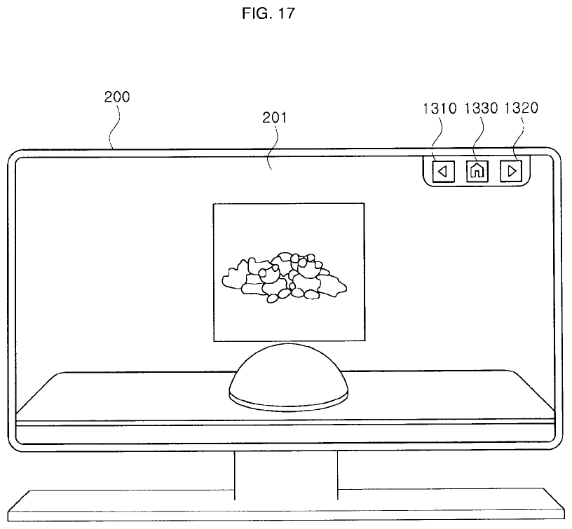

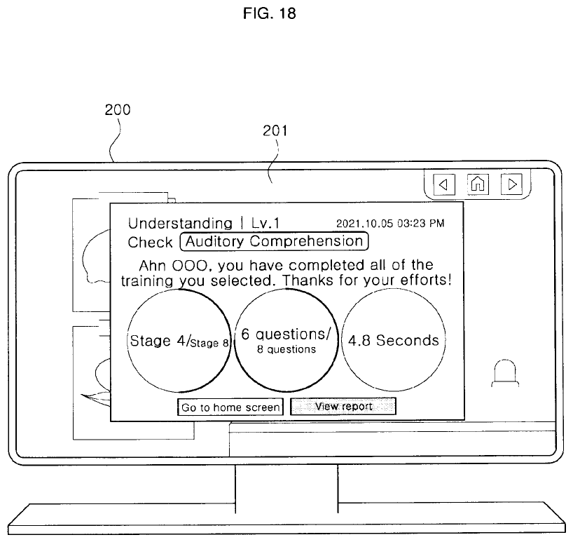

FIG. 17 is a diagram illustrating an example of a screen displayed to enable training to be performed through a display unit on a patient terminal according to an embodiment of the present disclosure, and FIG. 18 is a diagram illustrating an example of a screen displayed to enable confirmation of training result through a display unit on a patient terminal according to an embodiment of the present disclosure. Here, the screen displayed to enable training to be performed is a screen that appears as training begins, and the screen displayed to enable confirmation of training result is a screen that appears when training ends, that is, all training is completed.

Referring to FIG. 17, the training content provided to the patient may move to a previous problem 1310 and a next problem 1320, and the training may also be ended with an exit training 1330 button. When all training given through this screen is completed, a training completion screen may be provided as shown in FIG. 18. Referring to FIG. 18, the training results may be briefly provided, while buttons for ‘Go to home screen’ and ‘View report’ may be provided.

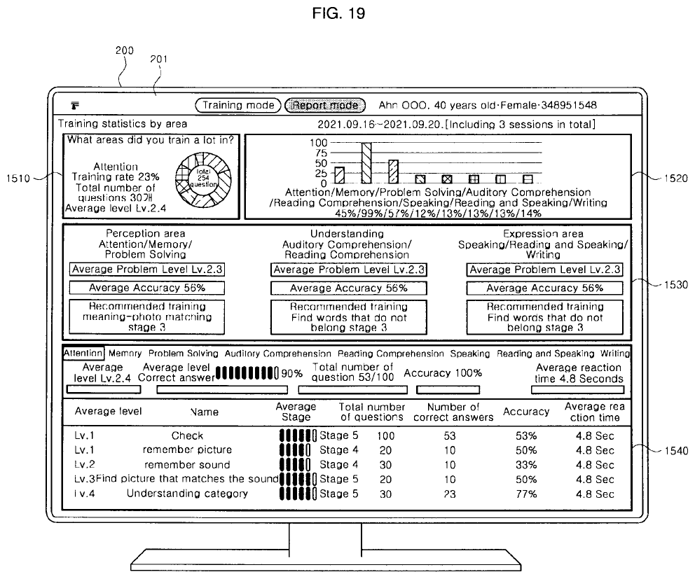

FIG. 19 is a diagram illustrating an example of a screen where analysis information according to performing training is displayed through a display unit on a patient terminal according to an embodiment of the present disclosure, and the screen may be a screen that appears by pressing the ‘View report’ by checking, touching, or clicking.

Referring to FIG. 19, a result 1510 of inquiring the patient's training area ratio and area data is provided, and an accuracy result 1520 for each detailed area is provided. In this case, the result 1510 of inquiring the patient's training area ratio and area data may visualize cumulative data of the learning date as a donut graph for each area, provide notation result for eight notation areas (attention, memory, problem solving, auditory comprehension, reading comprehension, speaking, reading, speaking, and writing), and provide area data inquiry result of area name/training ratio/total number of problems/average level. In addition, the accuracy result 1520 for each detailed area may provide a bar graph showing a correct answer rate for each learned area on the date of inquiry and the accuracy (y-axis) for eight areas (x-axis). In addition, the present disclosure provides a summary result 1530 for each training area, provides a summary box summarizing each of the three areas of cognitive rehabilitation category, comprehension rehabilitation category, and expression rehabilitation category, and calculation of an average level of all training problems, and displays the percentage of correct answers compared to the entire training. In addition, the present disclosure provides a detailed data inquiry result 1540 and shows training histories for the content in the corresponding area. For example, the present disclosure may provide information related to an average level, a name, an average stage, a total number of question, the number of correct answer, an accuracy, and an average reaction time.

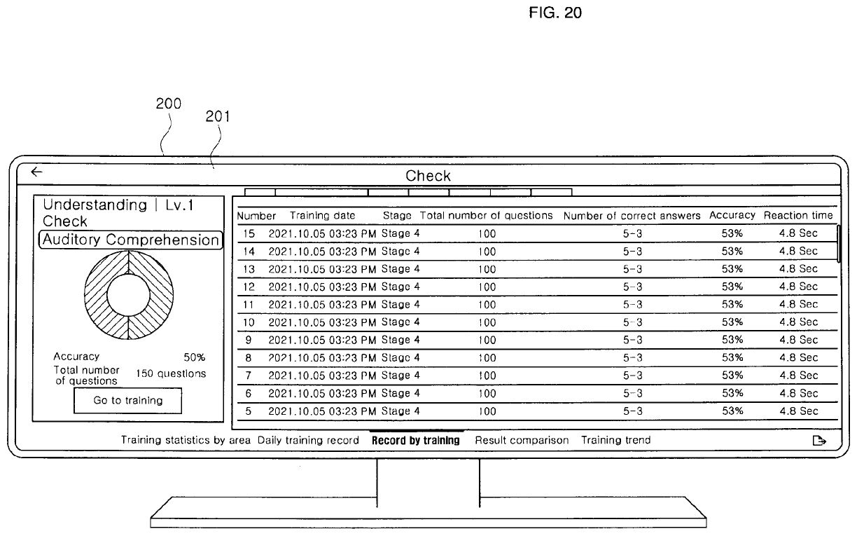

FIG. 20 is a diagram illustrating an example of a screen on which training performance history for a specific patient is displayed through a display unit on a patient terminal according to an embodiment of the present disclosure.

Referring to FIG. 20, the accuracy (correct answer rate) compared to the problem included in each training content is provided as a quantified graph, and ‘Go to training button’ may be provided to perform training by moving to the screen for training settings for the problem (FIG. 16).

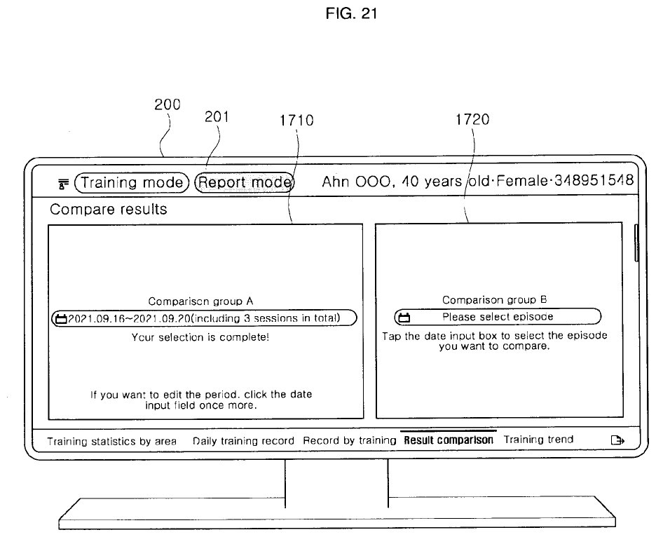

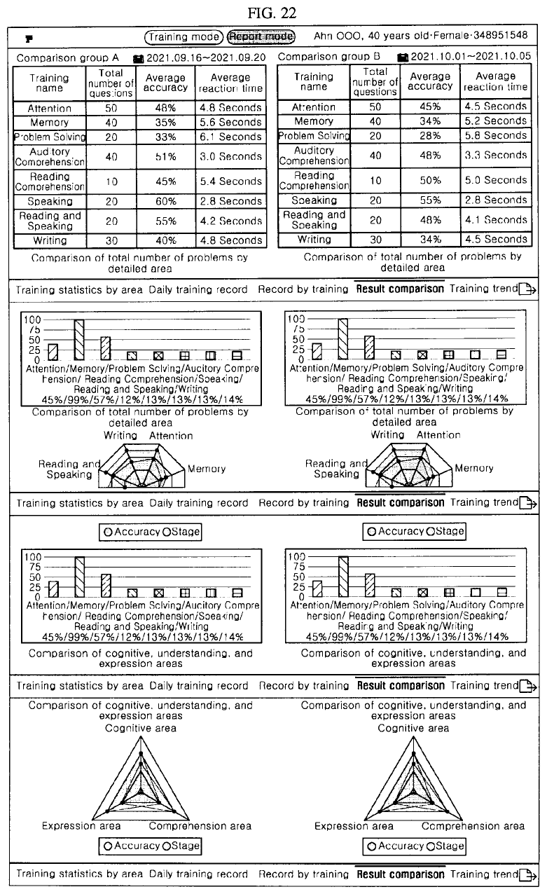

FIG. 21 is a diagram illustrating an example of a screen in which training results performed by a specific patient on different days are compared and displayed through a display unit on a patient terminal according to an embodiment of the present disclosure, and FIG. 22 is a diagram illustrating an example of each result report as a result of training performed on different dates according to an embodiment of the present disclosure.

First, referring to FIG. 21, a result comparison screen 1710 before date selection and a result comparison screen 1720 after date selection are provided to visually identify the results of improved language training by allowing the patient to perform the training content.

Meanwhile, referring to FIG. 22, each result report may be provided in the result comparison screen 1710 before date selection and the result comparison screen 1720 after date selection. For example, the result data of training name/total number of problems/average accuracy/average reaction time are provided in comparison group A and B periods, and total number of problems by area, accuracy and stage comparison by detailed area, and average reaction time by area, and recognition/understanding/expression area accuracy and stage comparison results may be provided in graphs.

Although FIG. 22 does not show a screen displayed on the display unit 201 of the patient terminal 200, this may also be displayed and provided on the display unit 201 of the patient terminal 200.

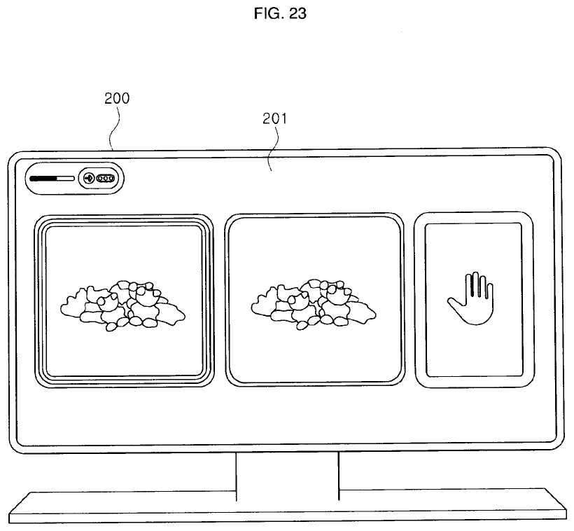

Meanwhile, the training performed through the present disclosure may be classified into the areas of attention, memory, executive power (executive function), comprehension (perceptual function), and language ability (language function). Hereinafter, the training in each area will be described based on FIGS. 23 to 29.

FIG. 23 is a diagram illustrating an example of a screen in which content for performing attention training through a patient terminal is displayed through a display unit on a patient terminal according to an embodiment of the present disclosure.

First, attention may be classified into sub-areas of sustained attention (the mental function of concentrating for the required amount of time), selective attention (the mental function of selecting competing internal or external stimuli and focusing on the selected stimulus), transitional attention (the mental function of focusing on one stimulus and focusing another stimulus again), and divided attention (the mental function to focus on two or more stimuli simultaneously) and divided attention (the mental function to focus on two or more stimuli simultaneously).

For example, attention is a means of limiting the amount of information that we actually process by focusing on the information momentarily among the many pieces of information that come in through eyes and ears, which are organs that receive important information from the outside. Training related to attention may include subcategories such as ‘reacting’, ‘identifying the same picture’, ‘following patterns’, ‘calculating’, and ‘counting’.

Referring to FIG. 23, this corresponds to ‘identifying the same picture’ among the training related to attention, and this training involves recognizing visual stimulus (picture) and maintaining attention to respond to the same visual stimulus (picture).

For example, in this process, setting the training stage, number of problems, and presentation speed are performed, and after checking the picture, when a picture similar to the example picture appears, pressing is performed. Whether the answer is correct or incorrect is automatically determined.

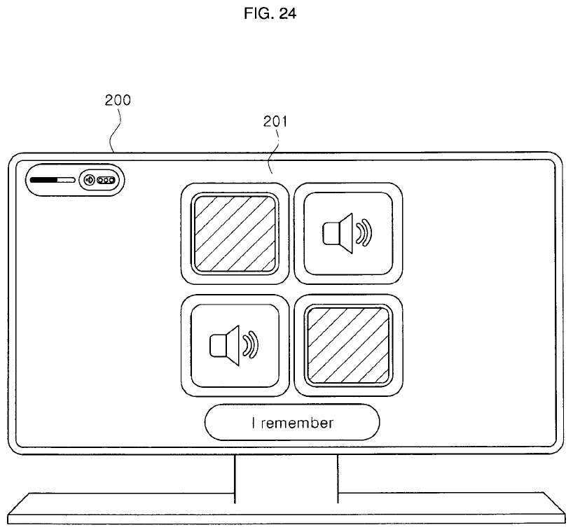

FIG. 24 is a diagram illustrating an example of a screen in which content for performing memory training through a patient terminal is displayed through a display unit on a patient terminal according to an embodiment of the present disclosure.

First, memory may be classified into sub-areas of short-term memory (a temporary and easily lost memory stored for about 30 seconds, the mental function that is lost if not strengthened into long-term memory), long-term memory (the mental function that creates a memory system that stores short-term memory, autobiographical memory, which is stored information about past events, and semantic memory, which is stored information about language and facts, for a long period of time) and working memory (the mental function that compares and processes information recalled from both short-term memory and long-term memory).

For example, memory is a series of processes that involve registering information, retaining or storing that information, and recalling or retrieving it over time. It involves not only receiving information, but also processing it, and combining received information. Recall means recalling stored information in a specific situation. Training related to memory may include subcategories such as ‘remembering the same picture’, ‘remembering the same sound’, ‘remembering the location’, ‘remembering the word’, and ‘remembering the number’.

Referring to FIG. 24, this corresponds to ‘remembering the same sound’ among memory-related training. This training is training to recognize auditory stimulus (sound) and remember the positional relationship between the same stimuli.

For example, in this state, it is performed setting the training stage, number of problems, and presentation speed, selecting an overturned card to check all the sounds, and then selecting the location of the memorized sound and matching it. Whether the answer is correct or incorrect is automatically determined.

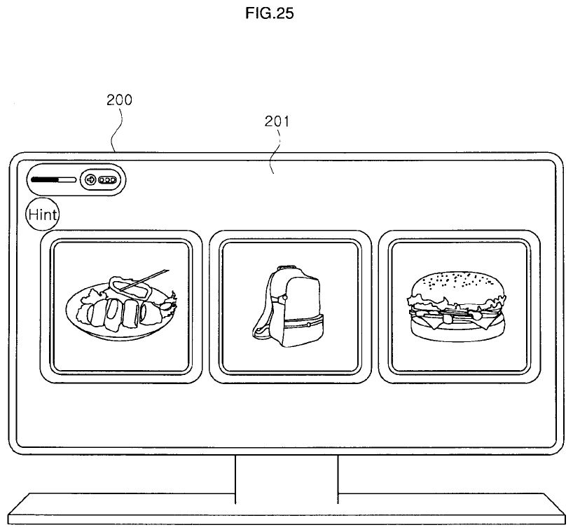

FIG. 25 is a diagram illustrating an example of a screen in which content for performing executive ability training through a patient terminal is displayed through a display unit on a patient terminal according to an embodiment of the present disclosure.

First, executive ability may be classified into sub-areas of abstraction (the mental function of creating general concepts, characteristics, or characteristics that are distinct from concrete facts, specific objects, or actual situations), organizing, and planning (the mental function involved in organizing and systematizing parts into a whole and the mental function involved in developing ways of proceeding or executing), cognitive flexibility (the mental function involved in problem-solving, changing strategies or shifting mental states), and judgment (the mental function involved in distinguishing and engaging different selective matters such as the function involved in forming opinions), and problem-solving ability (the mental function that identifies, analyzes and integrates inappropriate or contradictory information to find a solution).

For example, executive ability includes cognitive strategies such as understanding numbers, symbols, and formulas, arithmetic skills such as addition, subtraction, multiplication, and division, reasoning, understanding, and solving problems related to numbers, and processing, making inferences, and inferences of information. Training related to executive ability may include sub-categories such as ‘calculating money’, ‘finding something different’, ‘categorizing’, ‘finding rule’, and ‘decoding symbol’.

Referring to FIG. 25, this corresponds to ‘finding something different’ among the training related to executive ability, and this training is training to recognize and interpret a visual stimulus (picture) to abstract and judge the relationships between stimuli.

For example, setting the training stage, number of problems, and time limit, checking the presented pictures, and then selecting one picture that corresponds to another among the pictures are performed. Whether the answer is correct or incorrect is automatically determined.

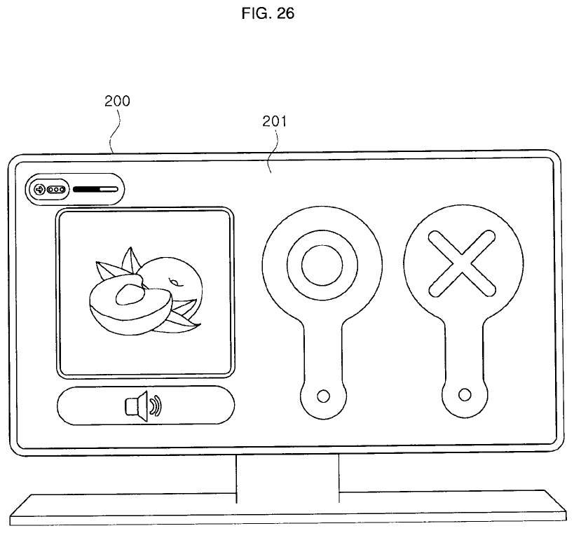

FIGS. 26 to 29 are diagrams illustrating a screen in which content for performing training for each of listening (auditory comprehension), speaking, reading, and writing is displayed through a patient terminal according to an embodiment of the present disclosure.

Language ability may be classified into sub-areas of voice language understanding (the mental function to interpret voice messages and understand their meaning), voice language expression (the mental function required to create meaningful voice messages), text language understanding (the mental function to interpret and understand text messages), text language expression (mental function necessary to create meaningful text messages).

In other words, language ability may be divided into speaking, listening, reading, and writing. Listening (auditory comprehension) belongs to the area of language understanding, and this ability is used to hear and understand messages in oral communication situations and communicate smoothly. Training related to listening (auditory comprehension) may include ‘selecting O/X (picture)’, ‘selecting O/X (word)’, ‘auditory word recognition’, ‘finding the correct answer in the picture (listening)’, ‘matching meaning-picture (listening)’, ‘listening and arranging words’, and ‘following instruction (listening)’.

Referring to FIG. 26, this corresponds to ‘Select O/X’ among the training related to listening (auditory comprehension). This training interprets visual stimuli (pictures) and auditory stimuli (words) to understand and compare meanings.

For example, setting the training stage, number of problems, and time limit, pressing the sound button to hear the voice, and then selecting O or X by comparing the meaning of the stimulus are performed. Whether the answer is correct or incorrect is automatically determined.

Meanwhile, speaking belongs to the area of language expression, and this ability is the ability to communicate smoothly by expressing messages in oral communication situations. Nonverbal communication, such as speed, pitch, volume, and voice characteristics, has a significant impact on interpreting what is being said. Training related to speaking may include sub-categories such as ‘practicing rhyme’, ‘practicing automatic spoken language’, ‘speaking after word’, ‘speaking after non-word’, ‘saying name’, ‘speaking after number’, ‘listening to explanation and saying correct answer’, ‘speaking after sentence’, ‘associating word’, ‘explaining’, and ‘combining words (speaking)’.

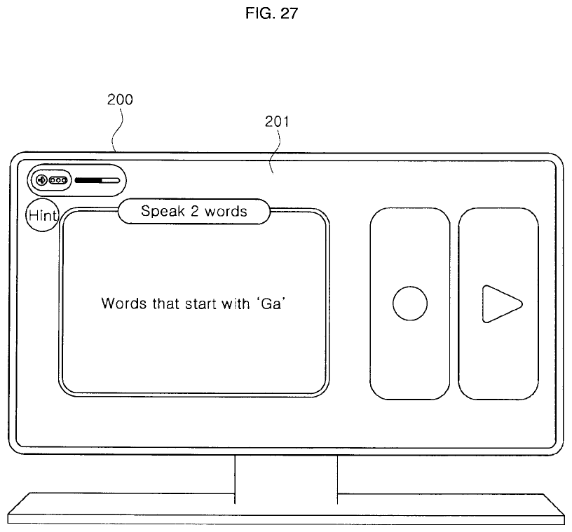

Referring to FIG. 27, this corresponds to ‘associating word’ among the training related to speaking. This training involves interpreting visual stimuli (presentation word) to understand meaning, infer and judge, and convert them into vocal expression (word).

For example, setting the training stage, number of questions, and time limit, checking the suggested word, pressing the record button to say the correct answer, and pressing the play button to confirm are performed. Whether the answer is correct or incorrect is automatically determined.

In addition, reading refers to reading fluency, that is, the activity of reading aloud in words that are explained by reading aloud, at exactly the right speed, and with appropriate intonation and expression. Training related to reading may include subcategory areas such as ‘matching picture-word’, ‘matching word-picture’, filling in blank’, ‘finding correct answer in the picture (reading)’, ‘reading number’, ‘reading non-word’, ‘matching picture (reading)’, ‘matching meaning-word (reading)’, ‘reading sentence’, and ‘following instruction (reading)’.

Referring to FIG. 28, this corresponds to ‘reading word’ among the training related to reading, and this training is training to interpret visual stimuli (words) and convert them into vocal expressions (words).

For example, setting the training stage, number of problems, and time limit, check the word, pressing the record button to say the word, and pressing the play button to confirm are performed. Whether the answer is correct or incorrect is automatically determined.

Meanwhile, writing is a complex language domain and is based on listening, speaking, and reading skills. Writing includes writing letter, spelling, and composition. The ultimate goal is writing, and to achieve this, correct spelling and the executive functions required in the writing process are necessary. Training related to writing may include subcategories such as ‘writing the same way’, ‘writing in the blank’, ‘writing the correct answer after looking at a picture’, ‘dictation’, and ‘combining words (writing)’.

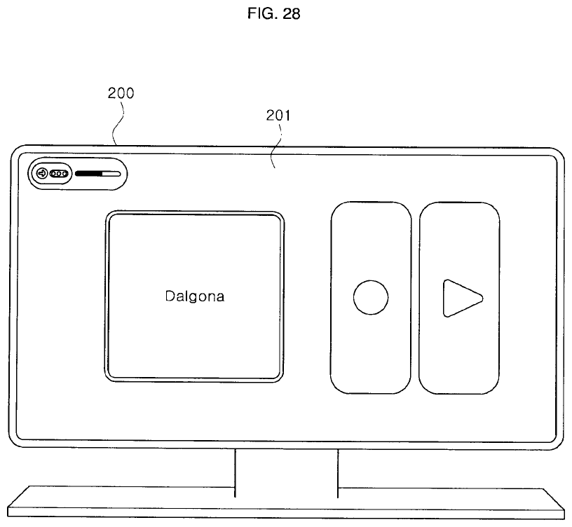

Referring to FIG. 29, this corresponds to ‘writing the same way’ among the training related to writing, and this training is training to interpret visual stimuli (words) and convert them into body movements (writing).

For example, setting the training stage, number of problems, and time limit, and pressing the write button to write the words in the same way (all entered content may be erased with the eraser button). Whether the answer is correct or incorrect is automatically determined.

Previously, in describing FIGS. 23 to 29, the difficulty of each subcategory area may be set prior to training. For example, as the number of visual and/or auditory stimulating elements increases or the presentation speed and/or time limit becomes shorter, a higher level of difficulty is set.

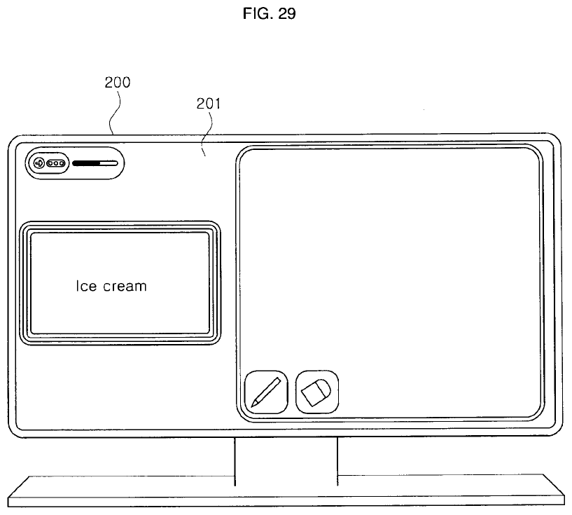

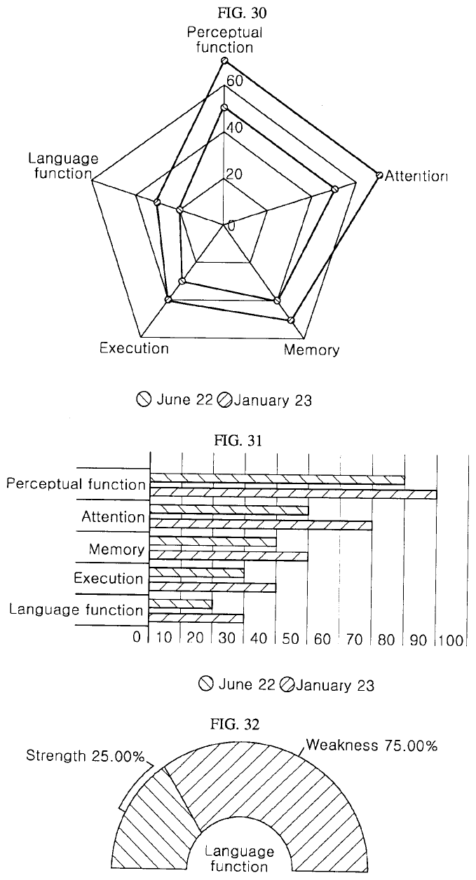

FIGS. 30 to 33 are diagrams illustrating an example of a graph generated according to an embodiment of the present disclosure.

Referring to FIGS. 30 and 31, the five upper regions including attention, memory, executive power (executive function), comprehension (perceptual function), and language ability (linguistic function) are configured, and a spider web chart as shown in FIG. 30 and/or, a horizontal bar chart as shown in FIG. 31 may be created. The score for each area is an overall score, which is calculated based on the scores of the subareas (average score).

At this time, scores for each of the areas of attention, memory, executive power (executive function), comprehension (perceptual function), and language ability (language function) may be calculated based on the scoring system as represented in Table 1 below.

TABLE 1

| | | | Damage level | |
|---|---|---|---|---|
| Percentage | ICF score | | (graded) | Examples |
| 96-100% | 4 | points | COMPLETE | Stage 0 |
| | | | impairment | (stage 1 fail) |
| 50-95% | 3 | points | SEVERE | Stage 1 |
| | | | impairment | (stage 1 pass/stage 2 fail) |
| 25-49% | 2 | points | MODERATE | Stage 2 |
| | | | impairment | (stage 2 pass/stage 3 fail) |
| 5-24% | 1 | point | MILD | Stage 3 |
| | | | impairment | (stage 3 pass/stage 4 fail) |
| 0-4% | 0 | point | NO | Stage 4 |
| | | | impairment | (stage 4 pass) |

A spider web chart, a semicircle chart, and/or a horizontal bar chart may be created using the scores for each area calculated in this way.

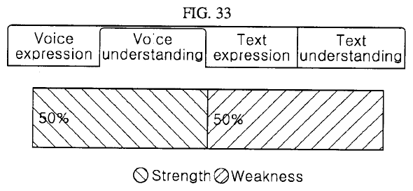

Meanwhile, referring to FIGS. 32 and 33, this is a score that may be viewed by selecting the upper region, and is composed of lower regions of each of the five upper regions to form a semicircle chart as shown in FIG. 32 and/or a progress chart as shown in FIG. 33 may be created. At this time, the standard score (Z-score) or inverse efficiency score (IES) of accuracy and reaction time may be calculated based on the Pass/Fail scoring system.

In this case, the standard score is calculated for accuracy and reaction time using Equation 1 below, and the ranking is analyzed using the two scores.

$$\begin{matrix}( Accuracy - Average Accuracy ) / Standard Deviation \\ ( Average reaction time - Reaction time ) / Standard Deviation\end{matrix} \qquad [ Equation 1 ]$$

Meanwhile, the reaction time of the task according to each content has a speed-accuracy exchange effect. According to the speed-accuracy exchange effect, if a subject responds quickly, the number of incorrect responses increases, and if the subject responds slowly, the number of correct responses increases. In other words, since more processing time is required for an accurate reaction, the reaction time is inevitably delayed.

The reaction time is converted and compared to inverse efficiency scores (IES) (Townsend & Ashby, 1983), which are modified values calculated by considering whether there is a forward reaction. The inverse efficiency score (IES) is a value obtained by dividing the reaction time (RT) by the ‘1-error response rate’, and may be calculated using Equation 2 below.

$$I E S = R T / P C \qquad [ Equation 2 ]$$

Here, RT represents the reaction time of correct response, and PC represents the proportion of correct response.

Meanwhile, as an additional possible analysis method, a first analysis method using perseverative responses, a second analysis method using trails to complete first task, a third analysis method using failure to maintain, and a fourth analysis method using unilateral neglect may be used.

Specifically, the first analysis method is to measure the number of times of repeating the same behavior while not being corrected (e.g., failing to learn and adapt to new things such as touching the same area repeatedly, continuing to select the same answer as the previous answer, etc.), and the second analysis method is to measure the process of learning and application through repeated failures (e.g., the number of attempts to answer the first step of remembering numbers).

In addition, the third analysis method is to measure the process from continuously getting the correct answer (e.g., 3 to 5 consecutive correct answers) to making an error (e.g., 3 consecutive errors after 5 consecutive correct responses in the fourth step of counting), and the fourth analysis method is to use the characteristic that correct responses are concentrated on the left or right, and there are standardized tests for this, such as star cancellation, albert test, and bells test (e.g., if the example presented in the right is the correct answer, a correct response is shown, but if the correct answer is on the left, the reaction speed is slowed or the answer on the right is selected and an incorrect reaction is shown).

So far, the present disclosure provides a service for training content that allows patients to perform various customized rehabilitation training at a desired time and place based on a cloud server, and enables continuous service improvement by reflecting opinions of a customer (patient, guardian, medical personnel, therapist, medical institution manager, etc.).

In addition, the present disclosure provides customized content for rehabilitation training related to at least one or more areas of memory, executive power, attention, auditory comprehension, speech, reading, and writing, and according to the present disclosure, a patient may receive customized speech rehabilitation treatment without spatial and time constraints, and a guardian and/or a medical personnel (doctor, nurse, rehabilitator, etc.) may monitor the patient's speech rehabilitation training process, results, and status.

In addition, according to the present disclosure, content that enables rehabilitation training tailored to the patient's level, such as stage (level), speed, and time, is provided, and analysis information is provided by analyzing the training pattern and/or training history. This allows the patient, as well as a guardian and medical personnel, to easily check the training result, and record them cumulatively to continuously manage the patient's condition according to the rehabilitation training.

Meanwhile, the disclosed embodiments may be implemented in the form of a recording medium that stores instructions executable by a computer. Instructions may be stored in the form of program code, and when executed by a processor, may create program modules to perform operations of the disclosed embodiments. The recording medium may be implemented as a computer-readable recording medium.

The computer-readable recording medium includes all types of recording media storing instructions that may be decoded by a computer. For example, there may be Read Only Memory (ROM), Random Access Memory (RAM), magnetic tape, magnetic disk, flash memory, optical data storage device, and the like.

As described above, the disclosed embodiments have been described with reference to the attached drawings. A person skilled in the art to which this disclosure pertains will understand that the present disclosure may be practiced in forms different from the disclosed embodiments without changing the technical idea or essential features of the present disclosure. The disclosed embodiments are illustrative and should not be construed as limiting.

What is claimed is:

1. A method for providing content for cognitive or language rehabilitation training based on a cloud server, performed by an apparatus, the method comprising:
upon detecting a login of a specific account into a platform linked to the cloud server, identifying a permission of the specific account;
as a result of the identification, based on the specific account being a patient account, collecting at least one medical information for the patient input from at least one connection account linked to the patient account;
generating status information of the patient by analyzing the at least one medical information;
searching at least one content based on the status information using an artificial intelligence-based learning model; and
generating and providing training information using the searched at least one content,
wherein the at least one connection account includes at least one of a guardian account, a therapist account, a medical personnel account, or a medical institution administrator account,
wherein the at least one medical information includes at least one of symptom information, diagnosis information, medical personnel information, therapist information, environmental information, or survey response information for the patient, and
wherein the status information represents the status of the patient as a quantitative indicator.

2. The method of claim 1, wherein searching the at least one content includes:
identifying a type of the content based on tag information of pre-stored contents based on the cloud server through a big-data analysis, and searching the at least one content based on the identified type of the content.

3. The method of claim 2, further comprising:
after generating and providing the training information,
upon receiving training result information generated as the patient performs training based on the training information, generating analysis information for the patient by analyzing the training result information; and
transmitting the analysis information to at least one preset terminal.

4. The method of claim 3, wherein, in the training result information, a degree of training performance, training ratio, training area, and accuracy for each training area for cognitive and language areas are recorded by date, and
wherein, when a user of the at least one terminal sets a date section, the analysis information is generated using the training result information included in the set date section and displayed as at least one of a graph and a number through a display of the at least one terminal.

5. The method of claim 2, wherein generating the status information includes:
evaluating at least one of a cognitive level or a language level of the patient using the quantitative indicator;
determining a training difficulty for the patient according to the evaluation result;
selecting the at least one content corresponding to the determined training difficulty among the at least one searched content; and
determining a training order based on the selected at least one content.

6. The method of claim 3,
wherein the at least one preset terminal is includes at least one of a patient terminal of the patient, a medical personnel terminal for each of at least one medical personnel, a therapist terminal for each of at least one therapist, a guardian terminal for each of at least one guardian, or a terminal provided in each of at least one medical institution,
wherein a permission of the patient information is set differently for each of the patient, the at least one medical personnel, the at least one therapist, the at least one guardian, or the at least one medical institution, and
wherein the analysis information is generated differently depending on the permission which is set.

7. The method of claim 6,
wherein, upon receiving request information requesting to provide the training information to the patient terminal, a payment procedure is performed, and
wherein the payment procedure is performed automatically based on pre-registered payment information or performed manually.

8. The method of claim 2,
wherein the pre-stored contents include a cognitive therapy content, a first speech therapy content, and a second speech therapy content,
wherein the first speech therapy content is content that is linked to cognitive therapy and usable without a therapist, and
wherein the second speech therapy content is content performed offline or online through a therapist.

9. The method of claim 8,
wherein the training information is transmitted to a terminal where a login is performed,
wherein, when generating the above training information, the training information is generated differently depending on a type of terminal on which the login is performed.

10. An apparatus for providing content for cognitive or language rehabilitation training based on a cloud server, comprising:
a communication module;
a storage module configured to store various information for at least one patient, an artificial intelligence-based learning model for providing content for the cognitive or language rehabilitation training, and at least one process; and
a control module configured to perform an operation to provide content for the cognitive or language rehabilitation training based on the at least one process,
wherein the control muddle is configured to:
upon detecting a login of a specific account into a platform linked to the cloud server, identify a permission of the specific account, as a result of the identification, based on the specific account being a patient account, collect at least one medical information for the patient input from at least one connection account linked to the patient account, generate status information of the patient by analyzing the at least one medical information, search at least one content based on the status information using an artificial intelligence-based learning model, and generate and provide training information using the searched at least one content,
wherein the at least one connection account includes at least one of a guardian account, a therapist account, a medical personnel account, or a medical institution administrator account,
wherein the at least one medical information includes at least one of symptom information, diagnosis information, medical personnel information, therapist information, environmental information, or survey response information for the patient, and
wherein the status information represents the status of the patient as a quantitative indicator.

11. The apparatus of claim 1, wherein, when searching the at least one content,
the control module is configured to identify a type of the content based on tag information of pre-stored contents based on the cloud server through a big-data analysis, and search the at least one content based on the identified type of the content.

12. The apparatus of claim 11, wherein,
after generating and providing the training information, upon receiving training result information generated as the patient performs training based on the training information, the control module is configured to generate analysis information for the patient by analyzing the training result information, and transmit the analysis information to at least one preset terminal.

13. The apparatus of claim 11,
wherein the control module is configured to evaluate at least one of a cognitive level or a language level of the patient using the quantitative indicator, determine a training difficulty for the patient according to the evaluation result, select the at least one content corresponding to the determined training difficulty among the at least one searched content, and determine a training order based on the selected at least one content.

14. The apparatus of claim 11,
wherein the pre-stored contents include a cognitive therapy content, a first speech therapy content, and a second speech therapy content,
wherein the first speech therapy content is content that is linked to cognitive therapy and usable without a therapist, and
wherein the second speech therapy content is content performed offline or online through a therapist.

15. The apparatus of claim 14,
wherein the training information is transmitted to a terminal where a login is performed,
wherein, when generating the above training information, the training information is generated differently depending on a type of terminal on which the login is performed.
